(19) United States
(12) Patent Application Publication
(10) Pub. No.: US 2002/0018751 A1
(43) Pub. Date: Feb. 14, 2002

(54) CELLULAR AND SERUM PROTEIN ANCHORS FOR DIAGNOSTIC IMAGING

(76) Inventors: DOMINIQUE P. BRIDON, OUTREMONT (QUEBEC) (CA); DOMINIQUE BLANCHARD, NANTES (FR); ALAN M. EZRIN, MORAGA, CA (US); PHILLIPE POULETTY, ATHERTON, CA (US)

Correspondence Address: Michael R. Ward, Morrison & Foerster LLP, 425 Market street, San Francisco,, CA, 94105-2482, US

(21) Appl. No.: 09/327,764

(22) Filed: Jun. 7, 1999

Related U.S. Application Data
(63) Continuation-in-part of application No. 08/588,912, filed on Jan. 12, 1996, now abandoned.
(63) Continuation-in-part of application No. 08/477,900, filed on Jun. 7, 1995, now Pat. No. 6,103,233.
Application No. 08/477,900 is a continuation of application No. 08/237,346, filed on May 3, 1994, now Pat. No. 5,612,034.
Application No. 08/237,346 is a continuation-in-part of application No. 08/137,821, filed on Oct. 15, 1993, now abandoned.

(51) Int. Cl. A61K 49/00

(52) U.S. Cl. 424/9.1

(57) ABSTRACT

Compositions and methods of non-invasive diagnosis are provided. The imaging agents include a linking groups and a reactive entity capable of reaction with a reactive functionality to form a covalent bond therewith. The imaging agents may be in the form of a bifunctional anchor molecule. The bifunctional anchor molecules have a functional group capable of activation which, when activated, may form a covalent bond with a reactive functionality on a target protein present in the mammalian vascular system, thereby “anchoring” the molecule to that target protein. The bifunctional anchors are also conjugated, either directly or indirectly, to a diagnostic agent of interest which provides the ability to diagnostically and non-invasively image the mammalian vascular space. Vascular targets include both cellular- and noncellular-associated proteins present in the mammalian vascular system. The methods find use for numerous applications arising from the ability to diagnostically image the mammalian vascular space over an extended period of time or to preferentially diagnostically image only a specific cell type or compartment of the mammalian vascular space.

### RELATED APPLICATIONS

This application is a continuation-in-part of U.S. patent application Ser. No. 08/588,912 filed Jan. 12, 1996 and a continuation-in-part application of U.S. patent application Ser. No. 08/477,900 filed Jun. 7, 1995 which is a continuation application of U.S. application Ser. No. 08/237,346 filed May 3,1994 now U.S. Pat. No. 5,612,034 which is a continuation-in-part of U.S. application Ser. No. 08/137,821 filed Oct. 15, 1993, now abandoned.

### FIELD OF INVENTION

The field of this invention is in vivo diagnostic imaging of mammals.

### BACKGROUND OF THE INVENTION

It is frequently desirable to non-invasively image the mammalian vascular space for such purposes as detecting abnormalities in blood flow, measuring cardiac function or for visualizing anatomical structures of the circulatory system. For example, in the case of certain disorders of or injuries to the vascular system which affect blood flow, one may wish to detect and visualize abnormal bleeding or, alternatively, the presence of thromboses. One may also wish to measure the effect of certain vascular disorders on cardiac efficiency and ventricular output. Additionally, non-invasive diagnostic imaging of anatomical structures of the mammalian vascular system may allow for the early detection of developmental abnormalities or various lesions, e.g., tumors, associated with the vascular system.

Present methodologies for non-invasively imaging the mammalian vascular system include such diagnostic techniques as positron emission tomography (PET), computerized tomography (CT), single photon emission computerized tomography (SPECT), magnetic resonance imaging (MRI), nuclear magnetic imaging (NMI), fluoroscopy, ultrasound, etc. However, while these techniques are extremely useful for a variety of different applications, they often provide far less than the desired utility, particularly when one wishes to image the vascular space over an extended period of time.

There is, therefore, substantial interest in providing novel compositions and methods for enhancing the ability to image the mammalian vascular space over an extended period of time.

### SUMMARY OF THE INVENTION

Methods and compositions are provided for non-invasive imaging of mammals by employing modified diagnostic agents capable of covalently bonding to proteins in vivo. The methods and compositions allow for monitoring the mammalian vascular compartment over an extended period of time.

The reagent compositions of the present invention have the general formula X—Y—Z where X is a diagnostic imaging agent, Y is a linking group consisting of 0-30 atoms and Z is a reactive entity capable of reaction with a reactive functionality in vivo.

The diagnostic agent X may be a radioisotope of such elements as iodine (I), including ¹²³I, ¹²⁵I, ¹³¹I, etc., barium (Ba), gadolinium (Gd), technetium (Tc), including ⁹⁹Tc, phosphorus (P), including ³¹P, iron (Fe), manganese (Mn), thallium (TI), chromium (Cr), including Cr, carbon (C), including ¹⁴C, or the like. In the formula, Z is a reactive entity, such as carboxy, carboxy ester, where the ester group is of 1-8, more usually 1-6 carbon atoms, particularly a physiologically acceptable leaving group which activates the carboxy carbonyl for reaction with amino groups in an aqueous system, e.g. N-hydroxysuccinimide (NHS), N-hydroxysulfosuccinimide, (sulfo-NHS), maleimide-benzoyl-succinimide (MBS), gamma-maleimido-butyryloxy succinimide ester (GMBS) and maleimidopropionic acid (MPA), N-hydroxysuccinimide isocyanate, isothiocyanate, thiolester, thionocarboxylic acid ester, imino ester, mixed anhydride, e.g. carbodiimide anhydride, carbonate ester, etc. and the like. The reactive entity Z will covalently bond to reactive functionalities in vivo or ex vivo.

The composition X—Y—Z may be administered directly for in vivo imaging.

In a second format, the diagnostic imaging agents may be in the form of a bifunctional anchor molecule. In this format, the reagent composition may have the general formula: X—B—A—Y—Z wherein X is a diagnostic imaging agent, Y is a linker and Z is a reactive functionally, as discussed above. In the formula of the second format, A is the first binding member of a binding pair and B is seconding binding member of the binding pair. The binding members bind to each other non-covalently (binding).

The first binding member (A) is generally haptenic usually below about 1 KD and generally more than about 100 D. The first binding member will be a physiologically acceptable molecule which recognizes a reciprocal second binding member (B). The first binding member may be biotin where the second binding member would be avidin or streptavidin. An antibody binding site may be the first binding member while the antibody, particularly the Fab fragment, would be the second binding member.

In the second format, the molecule A—Y—Z may be administered to a mammal first. A—Y—Z will covalently bond in vivo to form A—Y—Z-protein. Next, the molecule X—B is administered so that first binding member A can bind in vivo to second binding member B to form X—B—A—Y—Z-protein. Imaging is then carried out by detection of X.

The applications of the subject invention encompass MRI, CT, PET, SPECT imaging, detection of blood flow, abnormal bleeding, thromboses and vascular inflammation, vessel imaging, measuring cardiac efficiency and/or visualizing anatomical structures of the circulatory system.

### DETAILED DESCRIPTION OF THE INVENTION

Definitions

To ensure a complete understanding of the invention the following definitions are provided:

Reactive Entities:Reactive entities are entities capable of forming a covalent bond. Such reactive agents are coupled or bonded to a diagnostic agent of interest. Reactive entities will generally be stable in an aqueous environment and will usually be carboxy, phosphoryl, or convenient acyl group, either as an ester or an anhydride, or an imidate, thereby capable of forming a covalent bond with an amino group at the target site to form an amide or amide derivative. For the most part, the esters will involve phenolic compounds, or be thiol esters, alkyl esters, phosphate esters, or the like.

While the reactive entity is usually chosen to react with an amino group at the target site, other reactive functionalities at the target site may be exploited. For example, the reactive functionality may comprise various phosphinyl or phosphonyl derivatives for the bonding to available hydroxyl functions at the target site or may comprise an imine, thioimine or disulfide for bonding to thiol residues.

Reactive Functionalities: The reactive functionalities available on proteins for covalent bond formation with the reactive group are primarily amino, carboxyl and thiol groups. While any of these may be used as the target for the reactive entity, for the most part, bonds to amino groups will be employed, particularly with the formation of amide bonds.

To form amide bonds, one may employ a wide variety of active carboxyl groups as the reactive functional group, particularly esters, where the hydroxyl group is physiologically acceptable at the levels required. While a number of different hydroxyl groups may be employed, the most convenient will be N-hydroxysuccinimide and N-hydroxy sulfosuccinimide, although other alcohols, which are functional in the vascular environment may also be employed. In some cases, special reagents find use such as diazo, azido, carbodiimide, an hydride, hydrazine, or thiol groups, depending on whether the reaction is in vivo or in vitro, the target, the specificity of the reactive entity, and the like.

Fixed blood components: Fixed blood components are non-mobile blood components and include tissues, membrane receptors, interstitial proteins, fibrin proteins, collagens, platelets, endothelial cells, epithelial cells and their associated membrane and membraneous receptors, somatic body cells, skeletal and smooth muscle cells, neuronal components, osteocytes and osteoclasts and all body tissues especially those associated with the circulatory and lymphatic systems.

Mobile blood components: Mobile blood components are blood components that do not have a fixed situs for any extended period of time, generally not exceeding five minutes, more usually one minute. Mobile blood components include soluble blood proteins such as immunoglobulins, serum albumin, ferritin, transferrin and the like.

Delivery Devices: Delivery devices are devices useful for local delivery of diagnostic agents. Delivery devices include catheters, syringes, trocars and endoscopes.

Protective Groups: Protective groups are chemical moieties utilized to protect reactive entities from reacting with themselves. Various protective groups are disclosed in U.S. Pat. No. 5,493,007 which is hereby incorporated by reference. Such protective groups include acetyl, fluorenylmethyloxycarbonyl (FMOC), t-butyloxy carbonyl (BOC), benzyloxycarbonyl (CBZ), and the like.

Linking Groups: Linking groups are chemical moieties that link or connect reactive entities to diagnostic agents. Linking groups may comprise one or more alkylene, alkyleneoxy, alkenylene, alkynylene or amino group substituted by alkyl groups; cycloalkylene groups, polycyclic groups, aryl groups, polyaryl groups, substituted aryl groups, heterocyclic groups, and substituted heterocyclic groups. Linking group will have from 2-100, more usually from 2-18, preferably from 6-12 atoms in the chain, particularly carbon, oxygen, phosphorous and nitrogen, more particularly carbon and oxygen.

Diagnostic Imaging Agents: Diagnostic imaging agents are agents useful in imaging the mammalian vascular system and include such agents as position emission tomography (PET) agents, computerized tomography (CT) agents, magnetic resonance imaging (MRI) agents, nuclear magnetic imaging agents (NMI), fluroscopy agents and ultrasound contrast agents.

Taking into account these definitions, in its first aspect, the invention is directed to diagnostic imaging agents which have been modified with reactive entities so that they will covalently react and bond in vivo with reactive functionalities on to a blood component and provide increased half lifes for the diagnostic agents.

The derivatized diagnostic agent of the present invention will, for the most part, have the following formula: X—Y—Z.

In the formula, X is a diagnostic agent selected from PET agent, CT agents, MRI agents, NMI agents, fluroscopy agents and ultrasound contrast agents. Diagnostic agents of interest include radioisotopes of such elements as iodine (I), including ¹²³I, ¹²⁵I, ¹³¹I, etc., barium (Ba), gadolinium (Gd), technetium (Tc), including ⁹⁹Tc, phosphorus (P), including ³¹P, iron (Fe), manganese (Mn), thallium (TI), chromium (Cr), including 51 Cr, carbon (C), including ¹⁴C, or the like, fluorescently labeled compounds, etc.

In the formula, Y is a linking group of from 0-30, more usually of from 2-12, preferably of from 4-12 atoms, particularly carbon, oxygen, phosphorous and nitrogen, more particularly carbon and oxygen, where the oxygen is preferably present as oxy ether, where Y may be alkylene, oxyalkylene, or polyoxyalkylene, where the oxyalkylene group has from 2-3 carbon atoms, and the like. A linking group of 0 atoms is preferred when it is desired to place X as close to Z as possible.

In the formula, Z is a reactive entity, such as carboxy, carboxy ester, where the ester group is of 1-8, more usually 1-6 carbon atoms, particularly a physiologically acceptable leaving group which activates the carboxy carbonyl for reaction with amino groups in an aqueous system, e.g. N-hydroxysuccinimide (NHS), N-hydroxysulfosuccinimide, (sulfo-NHS), maleimide-benzoyl-succinimide (MBS), gamma-maleimido-butyryloxy succinimide ester (GMBS) and maleimidopropionic acid (MPA), N-hydroxysuccinimide isocyanate, isothiocyanate, thiolester, thionocarboxylic acid ester, imino ester, mixed anhydride, e.g. carbodiimide anhydride, carbonate ester, etc. and the like. The reactive entity Z will covalently bond to reactive functionalities in vivo or ex vivo.

The reactive functionalities which are available on proteins for covalently bonding to the chemically reactive entity of the derivatized diagnostic agent are primarily amino groups, carboxyl groups and thiol groups. While any of these may be used as the target of the chemically reactive entity on the diagnostic agent, for the most part, bonds to amino groups will be employed, particularly with formation of amide bonds.

In an alternative embodiment the covalent bonding is achieved by administering to the vascular system of a mammalian host from one to two compounds, including at least a first compound comprising a bifunctional anchor molecule having an activated functional group capable of forming covalent bonds with reactive functionalities on a vascular protein or proteins, which is linked either to a diagnostic agent of interest or to a first binding member of a specific binding pair.

In this alternative embodiment, the diagnostic agent will generally have the formula A—Y—Z wherein Y is a linking group and Z is a reactive entity as described above. In this alternative embodiment A is a first binding member of a specific binding pair which specifically recognizes and binds (as opposed to covalently bonding to) a reciprocal second binding member, B.

In this alternative embodiment, by administering the first compound to the vascular system of a mammalian host (A—Y—Z), the activated functional group will covalently bond to reactive functionalities on a protein or proteins present in the vascular system, thereby creating a population of functionalized vascular proteins: A—Y—Z-Protein If the first compound comprises a bifunctional anchor molecule linked to a first binding member of a specific binding pair, a second compound comprising a reciprocal second binding member joined to a diagnostic agent of interest is administered to the vascular system at any time during the lifetime of the functionalized vascular proteins. (B—X). After administration, the second binding member will bind to the first binding member A—B, thereby anchoring the diagnostic agent of interest to the functionalized vascular protein or proteins: X—B—A—Y—Z-protein. Imaging of the diagnostic agent present in the host's vascular system can then be accomplished by traditional means.

Linker Molecules

In either format, (X—Y—Z) or (X—B—A—Y—Z) the linker (Y) is physiologically acceptable at utilized doses, stable in the vascular system and effectively presents the diagnostic agent of interest or first binding member in vivo. The linker may be aliphatic, alicyclic, aromatic or heterocyclic, or combinations thereof, and the selection will be primarily one of convenience. The linker may be substituted with heteroatoms including nitrogen, oxygen, sulfur, phosphorus. Groups which may be employed include alkylenes, arylenes, aralkylenes, cycloalkylenes, and the like.

Generally, the linker is from 1-30, usually 1-10, more usually of from 1-6 atoms in the chain, where the chain will include carbon and any of the heteroatoms indicated above. For the most part, the linker will be straight chain or cyclic, since there normally will be no benefit from side groups. The length of the linker will vary, particularly with the nature of the diagnostic agent of interest or the first binding member, since in some instances, the diagnostic agent of interest or the first binding member may have a chain or functionality associated with it. The length of the linker may be used to provide for flexibility, rigidity, polyfunctionality, orientation, or other characteristics for improved function of the bifunctional anchor molecule. The linker may also provide for preferential bonding to a given protein epitope or sequence present in the vasculature as compared to other proteins epitopes or sequences present in the vasculature.

A large number of small synthetic bifunctional organic compounds comprising an appropriate activatable or activated functional group are available for joining the activated functional group to the diagnostic agent of interest or to the first binding member of a specific binding pair. Illustrative compounds include: azidobenzoyl hydrazine, N-[4-(p-azidosalicylamino)butyl]-3′-[2′-pyridyidithio)propionamide, bis-sulfosuccinimidylsuberate, dimethyl adipimidate, disuccinimidyl tartrate, N-γ-maleimidobutyryloxysuccinimide ester, N-hydroxy sulfosuccinimidyl-4-azidobenzoate, N-succinimidyl [4-azidophynyl]-1,3′-dithiopropionate, N-succinimidyl [4-iodoacetyllaminobenzoate, glutaraldehyde, succinimidyl 4-[N-maleimidomethyl]cyclohexane-1-carboxylate.

The linker joining the activated functional group and the diagnostic agent of interest or the first binding member may be oligomeric in nature and possess a high non-covalent affinity for a specific protein present in the mammalian vasculature as compared to other proteins present in the mammalian vasculature. Such oligomeric anchor molecules allow one to direct diagnostic agents of interest to specific targets, cells and/or proteins, in the vasculature, thereby allowing one to preferentially enhance the diagnostic signal in a particular anatomical compartment.

Preferably, oligomeric anchor molecules which find use will have the capability of preferentially bonding to reactive functionalities on a specific protein present in the vascular system. Preferential bonding means that the anchor molecule exhibits some preferential bonding to the vascular protein of interest as against other proteins present in the vascular environment. The preference for bonding the specified protein target will normally be at least about 1.5, preferably at least about 2 times, and may be 5 times or more as compared to random bonding in the absence of the oligomer.

The oligomeric linker of the bifunctional anchor molecule may be an oligopeptide, oligonucleotide, combinations thereof, or the like. Generally, the number of monomeric units in an oligomeric linker will be from 4 to 12, more usually from 4 to 8 and preferably from 5 to 8. The monomer units may be naturally occurring or synthetic, generally being from about 2 to 30 carbon atoms, usually from about 2 to 18 carbon atoms and preferably from about 2 to 12 carbon atoms.

If the linker is an oligopeptide, the amino acid monomers may be naturally occurring or synthetic. Conveniently, the L-α-amino acids will be used, although the D-enantiomers may also be employed.

The amino acids employed will preferentially be free of reactive functionalities, particularly reactive functionalities which would react with the activated functional group or diagnostic agent of interest attached to the oligomeric linker. Therefore, the amino acids which are used will usually be free of reactive amino, guanidino and carboxy groups, frequently being free of hydroxy and thiol groups. Of particular interest are the naturally occurring amino acids having hydrocarbon side chains including alanine (A), glycine (G), proline (P), valine (V), phenylalanine (F), isoleucine (I) and leucine (L) or uncharged polar amino acids like methionine (M).

The amino acid monomers of an oligopeptide linker may also be synthetic. Thus, any unnatural or substituted amino acids of from 4 to 30, usually from 4 to 20, carbon atoms may be employed. Of particular interest are the synthetic amino acids β-alanine and γ-aminobutyrate or functional group protected amino acids such as 0-methyl-substituted threonine (T), serine (S), tyrosine (Y), or the like.

Amino acids which find use may have the carboxyl group at a site other than the terminal carbon atom, may have the amino group at a site other than the α-position or may be substituted with groups other than oxy, thio, carboxy, amino or guanidino.

Synthetic amino acids may also be monosubstituted on nitrogen. N-substituted amino acids which find use will have an N-substituent of from about 1 to 8, usually 1 to 6 carbon atoms, which may be aliphatic, alicyclic, aromatic or heterocyclic, usually having not more than about 3 heteroatoms, which may include amino, either tertiary or quaternary, oxy, thio, and the like.

Oligopeptide linkers are usually constructed by employing standard Merrifield solid phase synthetic techniques using an automated peptide synthesizer, standard protection chemistry (e.g., t-boc or f-moc chemistry) and resins (e.g., 4-methyl benzhydryl amine). Other synthetic techniques, however, such as liquid phase oligopeptide synthesis may also find use.

Once synthesized, an available functional group on the oligomeric linker may be activated so as to be able to covalently bond to a reactive functionality present on a vascular protein in the environment in which the reaction is to occur. The activated functional group may be present at any position on the oligomeric linker, but will usually be proximal to one or the other terminus. Conveniently, a member of the oligomer may carry the activated functional group, such as on an aspartate or glutamate moiety.

For activation of a carboxyl group on the oligomeric linker, one may use a wide variety of anhydride or ester leaving groups, where the leaving group may have oxygen or sulfur bonded to carbonyl. In instances where one is interested in using the oligomeric anchor molecule in vivo, one may select the leaving group to be physiologically acceptable. Compounds which may be used to activate the carboxyl functional group include carbodiimides, phenols, thiophenols, benzyl alcohols, N-hydroxy imides, etc.

If the oligomeric linker is synthesized on a solid support, a functional group on the oligomer may be activated and the activated oligomer subsequently liberated from the solid support. Alternatively, the oligomer may be liberated from the support, thereby providing an available functional group for activation, and the functional group subsequently activated.

Bifunctional anchor molecules, whether comprising an oligomeric or non-oligomeric linker, are also coupled, either directly or indirectly, to a diagnostic agent of interest which imparts the ability to diagnostically image the mammalian vascular space. Preferably, the diagnostic agent is such that it does not react with the activated functional group of the bifunctional anchor molecule nor is it affected when the functional group of the anchor molecule is activated. Thus, diagnostic agents which find use generally do not react with reactive carboxyl and amino groups.

Methods of Producing Diagnostic Agents

The manner of producing the diagnostic agents of the present invention will vary widely, depending upon the nature of the various elements comprising the molecule. The synthetic procedures will be selected so as to be simple, provide for high yields, and allow for a highly purified product. Normally, the chemically reactive group will be created as the last stage, for example, with a carboxyl group, esterification to form an active ester will be the last step of the synthesis. Methods for the production of the diagnostic agents of the present invention are described in Examples 1-7.

Methods of Imaging

The methods of imaging comprise covalently bonding a diagnostic agent of interest to a protein or proteins. The diagnostic agent may be bonded to the protein in vivo or ex vivo. In ex vivo applications, the diagnostic agents of this invention are covalently bonded to a protein ex vivo and the protein(s) are administered to the vascular system. In in vivo applications, the diagnostic agents are administered directly either locally or for sytstemic administration and the agents become bound. The agents may bond to one or more long lasting blood proteins. The protein may be fixed or mobile. Once bound the agents may allow one to diagnostically image the mammalian compartment space over an extended period of time. Alternatively, the diagnostic agent may be preferentially bound to a specific protein or limited number of proteins present in the vascular system, thereby enhancing the ability to diagnostically image only a specific compartment.

The labeled material may be examined in vivo as discussed above or the tissue may be removed and examined outside the body. In this format, the removed tissue sample may be analyzed by immunohistochemisty for the presence absence of the diagnostic imaging agent.

The diagnostic agents of the invention are administered directed in the form of a bolus or introduced slowly over time by infusion using metered flow or the like. For those diagnostic agents that are administered directly, the agents may be designed for specific or non-specific labeling of proteins in vivo or ex vivo.

Specific Labeling

The diagnostic agents of this invention may be designed to specifically react with thiol groups on mobile blood proteins. Such reaction is preferably established by covalent bonding of a maleimide link (e.g. prepared from GMBA, MPA or other maleimides) to a thiol group on a mobile blood protein such as albumin or IgG.

Under certain circumstances, specific labeling with maleimides offers several advantages over non-specific labeling of mobile proteins with groups such as NHS and sulfo-NHS. Thiol groups are less abundant in vivo than amino groups. Therefore, the maleimide diagnostic agent derivatives of this invention will covalently bond to fewer proteins. For example, in albumin (the most abundant blood protein) there is only a single thiol group. Thus, contrast agent-maleimide-albumin conjugates will tend to comprise approximately a 1:1 ratio of contrast-agent derivatives to albumin. In addition to albumin, IgG molecules (class II) also have free thiols. Since IgG molecules and serum albumin make up the majority of the soluble protein in blood they also make up the majority of the free thiol groups in blood that are available for maleimide-contrast agent derivatives.

Through controlled administration of maleimide-contrast agent in vivo, one can control the specific labeling of albumin and IgG in vivo. In typical administrations, 80-90% of the administered maleimide-contrast agent will label albumin and 10-20% will label IgG. Trace labeling of free thiols such as glutathione will also occur. Such specific labeling is preferred for in vivo use as it permits an accurate calculation of the estimated half-life of the administered agent.

In addition to providing controlled specific in vivo labeling, maleimide-contrast agents can provide specific labeling of serum albumin and IgG ex vivo. Such ex vivo labeling involves the addition of contrast agent-maleimide to blood, serum or saline solution containing serum albumin. Once modified ex vivo with contrast agent-maleimide, the blood, serum or saline solution can be readministered to the blood for in vivo diagnostic analysis.

In contrast to NHS-derivatives, maleimide-contrast agents are generally quite stable in the presence of aqueous solutions and in the presence of free amines. Since maleimide-contrast agents will only react with free thiols, protective groups are generally not necessary to prevent the maleimide-contrast agent from reacting with itself. In addition, the increased stability of the peptide permits the use of further purification steps such as HPLC to prepare highly purified products suitable for in vivo use. Lastly, the increased chemical stability provides a product with a longer shelf life.

Non-Specific Labeling

Bonds to amino groups will also be employed, particularly with the formation of amide bonds for non-specific labeling. To form such bonds, one may use as a chemically reactive group a wide variety of active carboxyl groups, particularly esters, where the hydroxyl moiety is physiologically acceptable at the levels required. While a number of different hydroxyl groups may be employed in these linking agents, the most convenient would be N-hydroxysuccinimide (NHS) and N-hydroxy-sulfosuccinimide (sulfo-NHS).

Other linking agents which may be utilized are described in U.S. Pat. No. 5,612,034, which is hereby incorporated herein.

The various sites with which the chemically reactive group of the subject non-specific contrast agent derivatives may react in vivo include tissues; organs eg. liver, heart, lung, spleen and kidneys; fixed or mobile proteins, cells, particularly red blood cells (erythrocytes) and platelets, and soluble proteins, such as immunoglobulins, including IgG and IgM, serum albumin, ferritin, steroid binding proteins, transferrin, thyroxin binding protein, α-2-macroglobulin, and the like. Those proteins with which the derivatized contrast agent react, which are not long-lived, will generally be eliminated from the host within about three days. The proteins indicated above (including the proteins of the cells) will remain at least three days, usually at least four days, and may remain five days or more (usually not exceeding 60 days, more usually not exceeding 30 days) particularly as to the half life, based on the concentration in the blood, as measured in from about 1-3 hours after administration.

Use of Bifunctional Anchors

Generally, it will be satisfactory to have the diagnostic agent of interest bonded directly to the anchor molecule, thereby doing away with the need to administer a second compound comprising a second binding member attached to the diagnostic agent of interest. However, in certain situations it may be preferable to attach the diagnostic agent of interest to the bifunctional anchor molecule (which is covalently bonded to a vascular protein or proteins) indirectly, i.e., through the association between a first binding member of a specific binding pair with its reciprocal second binding member. Such situations include where it is useful to covalently bond the bifunctional anchor to vascular proteins in vivo and then periodically administer small amounts of the second binding member/diagnostic agent complex which is cleared from the vascular system, e.g., for use of diagnostic agents which are toxic when maintained at continuously high concentrations in vivo.

When the diagnostic agent of interest is bonded to the bifunctional anchor molecule indirectly, the first binding member which is joined to the bifunctional anchor molecule will generally be a small molecule, where the molecule is likely to minimize any immune response. Thus, for the most part, the first binding member will be haptenic, usually below about 1 kD and generally more than about 100 D, preferably less than about 600 D. Any physiologically acceptable molecule may be employed, where there is a convenient reciprocal second binding member. Thus, of particular interest is biotin, where avidin or streptavidin may be the reciprocal second binding member, but other molecules such as metal chelates, molecules mimicking a natural epitope or receptor or antibody binding site, also may find use, where the reciprocal second binding member may be an antibody or a fragment thereof, particularly a Fab fragment, an enzyme, a naturally occurring receptor, or the like. Thus, the first binding member may be a ligand for a naturally occurring receptor, a substrate for an enzyme, or a hapten with a reciprocal receptor.

The first binding member will naturally be found at low concentration, if at all, in the host vascular system, so there will be little if any competition between the first binding member and naturally occurring compounds in the vascular system for binding to the reciprocal second binding member. The reciprocal second binding member is such that it should not bind to compounds which it may encounter in the vascular system of the host.

The reciprocal second binding member of the specific binding pair will be determined by the nature of the first binding member employed. As already indicated, the second binding member may take numerous forms, particularly as binding proteins, such as immunoglobulins or fragments thereof, particularly Fab, Fv, or the like, particularly monovalent fragments, naturally occurring receptors, such as surface membrane proteins, enzymes, other binding proteins, such as avidin or streptavidin, or the like. Generally, the affinity of the second binding member for its reciprocal first binding member will be at least about 10⁻⁶, more usually about 10⁻⁸, e.g., binding affinities normally observed for the binding of monoclonal antibodies to their specific binding entities. Of particular interest is avidin and streptavidin, although other receptors of particular interest include receptors for steroids, LH, TSH, FSH, or their agonists, as well as sialic acid and viral hemagglutinins, and superantigens. The second binding member will usually be a macromolecule, generally of at least about 5 kD, more usually of at least about 10 kD and usually less than about 160 kD, preferably less than about 80 kD, which may be mono- or divalent in binding sites, usually monovalent.

The first compound and, if required, the second compound, will usually be administered as a bolus, but may be introduced slowly over time by transfusion using metered flow, or the like. Alternatively, although less preferable, blood may be removed from the host, treated ex vivo, and returned to the host. The first and second compounds will be administered in a physiologically acceptable medium, e.g., deionized water, phosphate buffered saline, saline, mannitol, aqueous glucose, alcohol, vegetable oil, or the like. Usually, a single injection will be employed although more than one injection may be used, if desired. The first and second compounds may be administered by any convenient means, including syringe, catheter, or the like. The particular manner of administration will vary depending upon the amount to be administered, whether a single bolus, sequential, or continuous administration, or the like. Administration will be intravascular, where the site of introduction is not critical to this invention, preferably at a site where there is rapid blood flow, e.g., intravenously, peripheral or central vein. The intent is that the compound administered be effectively distributed in the vascular system so as to be able to react with target proteins therein.

The dosage of the compound will depend upon whether it comprises the diagnostic agent of interest and will, therefore, be dependent on the adverse effects of the diagnostic agent of interest, if any, the time necessary to reduce the unbound concentration of the agent present in the vascular system, the dosage necessary for successful diagnostic imaging, the indication being sought, the sensitivity of the diagnostic agent to destruction by vascular components, the route of administration, and the like. As necessary, the dosage of diagnostic agent may be determined empirically, initially using a small multiple of the dosage normally administered, and as greater experience is obtained, enhancing the dosage. Dosages will generally be in the range of 1 ng/Kg to 10 mg/Kg, usually being determined empirically in accordance with known ways, as provided for in preclinical and clinical studies.

The mobile or fixed protein or proteins chosen as targets for reaction with the bifunctional anchor molecule will depend upon the indication desired. Thus, depending upon the vascular target or targets chosen, the diagnostic agent can be bonded to long-lived proteins and dispersed substantially throughout the entire vasculature, in which case the indication of choice will be diagnostic imaging of the vascular system over an extended period of time, or preferentially localized to specific areas of the vascular system, in which case the indication of choice will be the preferential diagnostic imaging of a specific anatomic compartment, either with or without imaging over an extended period of time. The target may be fixed or mobile; that is substantially fixed in position, as in the case of endothelial cells, or mobile in the vascular system, i.e., not having a fixed situs for an extended period of time, generally not exceeding 5, more usually, one minute. Target cells and proteins may have a substantially uniform or variant distribution in the vascular system, where the target may preferentially localize or be concentrated in particular compartments, as compared to the vascular system or other anatomic compartments.

Use of the Diagnostic Agents of the Invention

The diagnostic agent employed and the vascular protein or proteins targeted will depend upon whether one wishes to diagnostically image the anatomic compartment over an extended period of time, whether one wishes to preferentially image only a specific cell type or compartment, or both. Applications for covalently bonding a diagnostic agent of interest to a long-lived vascular protein for diagnostic imaging of the vascular space over an extended period of time are numerous and include enhancing the ability to detect abnormalities in blood flow throughout the entire mammalian vascular system, including the detection of internal injury causing abnormal bleeding or, alternatively, the presence of thromboses. For example, one may wish to image the vascular space over an extended period of time to detect the effects of a particular treatment while they occur, i.e., detecting the disappearance of an embolism, the stoppage of internal bleeding, or the like.

Diagnostically imaging the vascular space over an extended period of time also allows for the detection of various diseases associated with the vascular system, i.e., such as arterial blockage in the heart. Thus, diagnostically imaging the vascular system over an extended period of time may be employed to non-invasively detect a consistently reduced blood flow to the heart. Such a method also provides a means for quantitatively measuring cardiac efficiency and ventricular output volume over an extended period of time, i.e., during extended periods of exercise, or the like.

Other applications for such a method arise from the ability to non-invasively visualize anatomical structures of the mammalian vascular system and the effects on those anatomical structures over time of the administration of various drugs, such as vasodilators, vasoconstrictors, or the like. Such may allow for the early detection of developmental vascular abnormalities, injuries, or the like.

Additional applications arising from the ability to diagnostically image the vascular space over an extended period of time include functional assessment of the cardiovascular system as routinely utilized in nuclear medicine for single measurements.

Applications for preferentially bonding a diagnostic agent of interest to a specific protein or proteins present in the vascular system so as to diagnostically image only a specific cell type or compartment are also numerous. For example, having the ability to preferentially direct a diagnostic agent of interest to a specific cell type in the vascular system can allow for the non-invasive and early detection of lesions or various tumors associated with the mammalian vascular system by directing the bifunctional anchor molecule to a tumor specific cell surface protein.

Additionally, diagnostic agents can be directed to cell surface proteins of specific cell types predominantly associated with specific anatomic compartments, allowing one to preferentially diagnostically image such compartments as lymph nodes, Peyer's patches, kidney glomeruli, liver, pancreas, tonsil, or any other organ to which mobile cells in the vasculature will migrate.

Other applications for preferentially diagnostic imaging a specific cell type or compartment of the vascular system include diagnosis and treatment of stenosis or plaque, vascular shunt reendothelialization or shunt failure due to tissue growths, or organ rejection due to tissue migration.

The diagnostic agents of the invetion may be delivered to a local site via a local delivery device. Delivery devices include catheters, syringes, trocars and endoscopes. Delivery of the agent to a local site allows imaging of the specific area of delivery. The agents that find particular use in localized delivery are the non-specific diagnostic agents such as NHS-derivatives.

This application will be better understood by reference to the following non-limiting examples.

### EXAMPLE 1

Preparation of Rhodamine NHS Ester

Rhodamine Green™-X, succinimidyl ester, hydrochloride mixed isomers is commercially available from Molecular Probes (Eugene Oregon) as illustrated below:

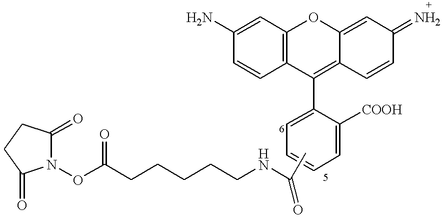

### EXAMPLE 2

In vivo Addition of NHS-rhodamine

New Zealand rabbits (2 Kg), male or female, were intramuscularly anesthetized with Xylazine (20 mg/kg), Ketamine (50 mg/kg) and Acepromazine (0.75 mg/kg) prior to surgical exposure of left carotid artery. Both carotid arteries were isolated and blood flows were measured. A catheter (22G) was inserted in the arterial segment and rinsed with 0.9% sodium chloride via catheter until there was no more visible evidence of blood in the segment.

A 1-cm incubation chamber was created by ligatures in the segment area. The incubation chamber was flushed three times with 1 mL of 0.9% sodium chloride. A solution of 100 μl of 500 μM NHS-Rhodamine was prepared and incubated in the incubation chamber for 3 minutes. The excess of rhodamine was withdrawn with a 1 mL syringue. The incubation chamber was washed once again with 3 times 100 mL of 0.9% sodium chloride. The incubation chamber was then removed from the rabbit, cut in three pieces and dipped in 10% formalin for further evaluation. The NHS-Rhodamine treated arteries exhibited dramatic levels of fluorescence whereas those arteries treated solely with Rhodamine exhibited little fluorescence over background. These results demonstrate that Rhodamine was covalently bonded to a local delivery site.

### EXAMPLE 3

Preparation of [³H]-NHS-propionate

[³H]-NHS-propionate is available from Amersham Canada Ltd. (Oakville, Ontario, Canada) and can be prepared from the tritiated propionic acid through known to the art condensation of N-hydrosuccinimide in presence of EDC in DMF or methylene chloride.

### EXAMPLE 4

In vivo Pharmacokinetics Studies of [³H]-NHS-propionate

New Zealand rabbits (2 kg), male or female, were intramuscularly anesthetized with Xylazine (20 mg/kg), Ketamine (50 mg/kg) et Acepromazine (0.75 mg/kg) prior to surgical exposure of left carotid artery. Segments of 10 mm of carotids, were transiently isolated by temporary ligatures and rinsed with 0.9% sodium chloride via a cannula until there was no more visible evidence of blood components.

A catheter (18G) was inserted in the arterial segment and served to introduce the angioplasty balloon (2.5 mm of diameter, over the wire/Boston Scientific Inc.). A vascular damage (angioplasty) was performed on the isolated segment in order to eliminate the layer of endothelial cells. The angioplasty balloon was serially inflated at different atmospheres (4, 6, 8 and 10) during 1 minute, with 45 seconds of delay between inflations. At 4 atmospheres a balloon traction was performed 5 times and 1000 U/kg of heparin were infused in the blood circulation.

The angioplasty balloon was then retrieved from the artery and the catheter was reintroduced. The arterial segment was rinsed 3 times with saline, and 100 μM of [³H ]-NHS-propionate was incubated within the isolated segment of the artery for either 30 seconds, 3 minutes or 30 minutes. At the end, the excess of incubation liquid was withdrawn from the artery, and the segment was rinsed 5 times with saline. The treated artery was immediately harvested, and incorporation of [³H]-labeled compounds within the artery was evaluated by scintillation counting. After 30 seconds of incubation, we recorded an association efficiency of 2.55%. At 3 min and 30 min, we recorded an association efficiency of 5.5 and 6.5%, respectively. We decided that a 3 min incubation time was sufficient to treat the artery in an efficient way.

When evaluating the retention levels, 100 μM of [³H ]-NHS-propionate or [³H ]-propionate were incubated with the artery for a period of 3 minutes, after which the segment has been rinsed 5 times with saline. The catheter was then removed and the arteriotomy site was closed with microsutures, thus reestablishing the blood flow within the carotid. Finally, the neck wound was closed with sutures, and animals are allowed to recuperate. Three days following the treatment, the animals are sacrificed with an overdose of sodium pentobarbital, the carotid segments are removed and examined for compound's presence by scintillation counting. 10.94% retention of [³H ]-NHS-propionate was monitored after three days following a 3 minute incubation period based on residual radioactivity in the artery. The difference in retention efficiency between covalently and non covalently bound propionate after a 3 minutes incubation period was determined. An outstanding 12 fold enhancement in retention was recorded (0.6% of total amount incubated against 0.046% for the non covalently bound) in favor of the NHS-propionate. This indicates that the tissue association of a compound is dramatically enhanced by the covalent attachment in vivo. Subsequent restitution of blood flow demonstrated retention [³H ]-NHS-propionate of approximately 10% of the material 72 hours after injury. This represents excessive tissue retention using the embodied technology of agents markedly beyond that seen with all drug delivery technologies as exemplified in the literature for standard non covalent agents (Circulation 1994 89 (4) 1518-1524).

### EXAMPLE 4

Synthesis of [³²P] NHS Derivative

To a solution of protected R and R′ (both R and R′ can be alkyl,

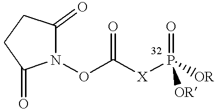

phenyl or alkoxy groups, and X is either O or S, alkoxy, alkyl and any other functionality stable under these conditions) phosphodiester (0.1 mmol) and N-hydroxysuccinimide (0.2 mmol) is added diisopropylethylamine 0.11 mmol), followed by addition of HBTU (0.22 mmol). The reaction mixture is stirred at room temperature for 36 hours. DMF is removed by vacuum distillation and the residue is dissolved in MeOH (10 mL). The MeOH solution is filtered to remove the insolubles, the filtrate is concentrated in vacuo, and the residue is dissolved in a minimum amount of MeOH. Water is then added to induce precipitation and the precipitate is dried on vacuum to give the desired compound.

The yield of the reaction can usually be improved by using EDC as the coupling reagent, as exemplified below. To a solution of R and R′ phosphodiester (0.054 mmol) and N-hydroxysuccinimide (0.115 mmol) in anhydrous DMF (3 mL), is added EDC (31 mg, 0.162 mmol). The solution is stirred at room temperature for 24 hours. DMF is removed by vacuum distillation and the residue is further dried on high vacuum. The residue is then dissolved in a minimum amount of MeOH (0.12 mL) and H₂O (3.2 mL) is added to induce precipitation. The precipitates are washed with H₂O (3×0.8 mL) and dried on vacuum to give a solid product.

Any protected phosphonate derivatives may undergo similar transformation.

### EXAMPLE 5

New Zealand rabbits (2 kg), male or female, were anesthetized with xylazine (20 mg/kg), ketamine (50 mg/kg) and acepromazine (0.75 mg/kg) intramuscularly prior to surgical exposure of left carotid artery. Carotid arteries were surgically dissected and segments of approximately 10 mm length were isolated. The vessels were cannulated and rinsed with 0.9% sodium chloride until there was no more visible evidence of blood components.

A catheter (18G) was inserted in the arterial segment and served to introduce the angioplasty balloon (2.5 mm of diameter, over the wire/Boston Scientific Inc.). Vascular damage (angioplasty) was performed on the isolated segment in order to eliminate the layer of endothelial cells. The angioplasty balloon was serially inflated at different atmospheres (4, 6, 8 and 10) for 1 minute, with 45 seconds of delay between inflations. At 4 atmospheres a balloon traction was performed 5 times and 1000 U/kg of heparin were infused in the blood circulation.

The angioplasty balloon was then retrieved from the artery and the catheter was reintroduced. The arterial segment was rinsed 3 times with saline, and 100 μM of [³²P]-NHS-[linker] was incubated within the isolated segment of the artery for 3 minutes. At the end, the excess of incubation liquid was withdrawn from the artery, and the segment was rinsed 5 times with saline. The vessel was sutured closed, blood flow restored and surgical wounds repaired. Animals were returned to the vivarium for periods up to four weeks. Tissue retention of [³²P]-NHS-[linker] was evaluating using whole animal radiography at selected periods of time after injury.

### EXAMPLE 6

Synthesis of ¹³¹I]-NHS Derivative

To a solution of protected amino protected [¹³¹ I]-iodotyrosine (0.1 mmol) and N-hydroxysuccinimide (0.2 mmol) is added diisopropylethylamine (0.11 mmol), followed by addition of HBTU (0.22 mmol). The reaction mixture is stirred at room temperature for 12 hours. DMF is removed by vacuum distillation and the residue is dissolved in MeOH (10 mL). The MeOH solution is filtered to remove the insolubles, the filtrate is concentrated in vacuo, and the residue is dissolved in a minimum amount of MeOH. Water is then added to induce precipitation and the precipitate is dried on vacuum to give the desired compound.

The yield of the reaction can usually be improved by using EDC as the coupling reagent, as exemplified below. To a solution of [¹³¹I]-iodotyrosine (0.054 mmol) and N-hydroxysuccinimide (0.115 mmol) in anhydrous DMF (3 mL), is added EDC (31 mg, 0.162 mmol). The solution is stirred at room temperature for 24 hours. DMF is removed by vacuum distillation and the residue is further dried on high vacuum. The residue is then dissolved in a minimum amount of MeOH (0.12 mL) and water (3.2 mL) is added to induce precipitation. The precipitates are washed with H₂O (3×0.8 mL) and dried on vacuum to give a solid product.

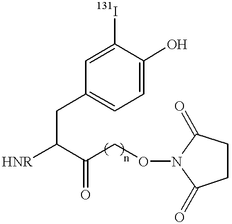

### EXAMPLE 7

In vivo Pharmacology of ¹³¹I Derivative

New Zealand rabbits (2 Kg), male or female, were anesthetized with xylazine (20 mg/kg), ketamine (50 mg/kg) and acepromazine (0.75 mg/kg) intramuscularly prior to surgical exposure of left carotid artery. Carotid arteries were surgically dissected and segments of approximately 10 mm length were isolated. The vessels were cannulated and rinsed with 0.9% sodium chloride until there was no more visible evidence of blood components.

A catheter (18G) was inserted in the arterial segment and served to introduce the angioplasty balloon (2.5 mm of diameter, over the wire/Boston Scientific Inc.). Vascular damage (angioplasty) was performed on the isolated segment in order to eliminate the layer of endothelial cells. The angioplasty balloon was serially inflated at different atmospheres (4, 6, 8 and 10) for 1 minute, with 45 seconds of delay between inflations. At 4 atmospheres a balloon traction was performed 5 times and 1000 U/kg of heparin were infused in the blood circulation.

The angioplasty balloon was then retrieved from the artery and the catheter was reintroduced. The arterial segment was rinsed 3 times with saline, and 100 μM of [¹³¹I]—NHS-[linker] was incubated within the isolated segment of the artery for 3 minutes. At the end, the excess of incubation liquid was withdrawn from the artery, and the segment was rinsed 5 times with saline. The vessel was sutured closed, blood flow restored and surgical wounds repaired. Animals were returned to the vivarium for periods up to four weeks. Tissue retention of [¹³¹I]-NHS-[linker] was evaluated using whole animal radiography at selected periods of time after injury.

### EXAMPLE 8

Binding of Biotin-NHS In vivo

5 mg (˜1.5 mg/kg) and 50 mg (˜15 mg/kg) of NHS-biotin solubilized in DMSO were injected into rabbits 3, or A and 8, respectively. Blood samples were then taken 0.5, 1, 2 and 4 h on the same day after injection and then on 1, 2, 3, 6, 9, 13, 20, 27, 34, 41, 48 and 55 days after injection.

30 minutes after injection, all RBCs were biotinylated as shown by flow cytometry performed with phycoerythrin-conjudated avidin. The mean fluorescence was much lower for rabbit 3 than for rabbit 8 (26 and 320, respectively), showing a direct relationship between the labeling of RBCs and the dose of NHS-biotin. Half-lives of 17 or 15 days were calculated from the curves obtained with rabbit 3 or 8, respectively. A life span of 55 days was obtained with rabbit 3. After 49 days, 6% of biotinylated RBCs survived for rabbit 8. As measured by conventional methods, the life span of rabbit RBCs is 45 to 70 days.

The plasma proteins of rabbits 3 and 8 were analysed by immunobloting. The plasma proteins were separated under reducing conditions (10 mM DTT) in a 10% polyacrylamide gel (Coomassie blue staining). Major components were identified as p180, p90, p75, albumin, and the heavy and light chains of immunoglobulins. All plasma proteins revealed with Ponceau red were able to capture avidin-phycoerythrin, showing that they are biotinylated. For rabbit 3, only serum albumin could be detected on day 20. The staining was much more intense with samples from rabbit 8, showing biotinylation of high molecular weight components (˜200 kDa) and the light chains of immunoglobulins. No staining was observed day 19.

The pattern of plasma proteins separated under non-reducing conditions in a 8% polyacrylamide gel (Coomassie blue staining) showed IgM, IgG, p90, p75, and serum albumin as the major components. On immunoblots, albumin (60 kDa), transferrin, p90, IgG (160 kDa) and high molecular weight components corresponding in part to IgM were detected. The half-life of molecular weight components and igG appeared longer than that of albumin, since the latter could not be visualized from samples taken day 12 from rabbit 8.

### EXAMPLE 9

Binding of Biotin-maleimide in vivo

23 mg of Biotin-maleimide (MW 451.5, SigmaChemical, St. Louis Mo., USA, Catalog number B-1267) was dissolved in 0.7 mL of DMSO and injected into a rabbit to give a final concentration of 500.M in 100 mL of blood. A similar injection was performed using similar volume of DMSO as a negative control.

Blood was taken immediately after injection at TO. Rabbits were sacrificed 50 minutes after injection and blood and main organs (kidneys, liver, lungs and spleen) were collected for further evaluation. Tissue samples were frozen in liquid nitrogen and kept at −80° C. before evaluation.

Plasma proteins were analyzed by immunoblotting. The plasma proteins were separated under reducing conditions (10 mM DTT) in a 10% polyacrylamide gel (Coomassie blue staining). Major components were identified as albumin and the heavy and light chains of immunoglobulins. Most of the other plasma proteins usually revealed with Ponceau red were not captured by the avidin-phycoerythin conjugate, showing absence of non selective biotinylation. The staining was much more intense for albumin as it was for both chains of immunoglobulins.

Immunohistological slides were prepared from the frozen tissue samples using a microtome Leica CM1900 by incubating the frozen cut tissue samples with the avidin-peroxidase complex (Biosys Vector) in PBS. Peroxidase substrate was then added to reveal the presence or absence of labeling from biotin.

The following labeling of tissue samples was obtained:

Spleen: very week labeling from the maleimide derivative. DMSO negative control showed no labeling at all.

Liver: Very week labeling from the maleimide derivative. Again the DMSO control showed no labeling.

Lung: very week labeling from the maleimide derivative. DMSO negative control showed no labeling at all.

Kidneys: very week labeling from the maleimide derivative. DMSO negative control showed detection of endogeneous biotin

To establish a comparison between biotin-NHS with Biotin-maleimide and to demonstrate the superior specificity of the latter, a similar amount of NHS-biotin (28 mg in 0.7 mL of DMSO) was injected into a rabbit and blood and tissue samples were collected after sacrifice of the animal. Immunohistological slides were prepared from the frozen tissue samples using a microtome Leica CM1900 by incubating the frozen cut tissue samples with the avidin-peroxydase complex (Biosys Vector) in PBS. Peroxidase substrate was then added to reveal the presence or absence of labeling from biotin.

The following labeling of tissue samples was obtained:

Spleen: intense labeling from the NHS derivative. DMSO negative control showed no labeling at all.

Liver: slight labeling from the NHS derivative. Again the DMSO control showed no labeling.

Lung: very intense labeling from the NHS derivative. DMSO negative control showed no labeling at all.

Kidneys: intense labeling from the NHS derivative. DMSO negative control showed detection of endogeneous biotin within the cortical area.

Analysis of all three rabbit's bloods by flow cytometry showed a very intense labeling of red blood cells from the biotin-NHS, a very weak labeling for the biotin-maleimide (signal close to noise level) and no labeling from DMSO alone demonstrating again the capability for biotin-NHS to label all blood components with high yield.

The results indicate that maleimide and NHS biotin derivatives injected in vivo are able to label blood components as well as biological tissue with various specificity. The results indicate that maleimide contrast agents by bonding selectively onto albumin should be used to perform diagnostic imaging of the vasculature. In contrast, NHS contrast agents should be used to perform non selective labeling of an area, for instance the labeling of an arterial wall post angioplasty, or the size reduction of a tumor after intratumoral injection of the diagnostic imaging agent via a catheter.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A composition comprising a compound of the formula:
X—Y—Z
wherein
X is a diagnostic imaging agent;
Y is a linking group consisting of 0-30 atoms; and
Z is a chemically reactive entity capable of reaction with a reactive functionality to form covalent bonds therewith.

2. The composition of claim 1 wherein said reactive functionality is selected from the group consisting of amino, carboxyl and thiol groups.

3. The composition of claim 1 wherein Z is selected from the group consisting of N-hydroxysuccinimide, N-hydroxy sulfosuccinimide, maleimide-benzoyl-succinimide, gamma-maleimido-butyryloxy succinimide ester, maleimidopropionic acid, isocyanate, thiolester, thionocarboxylic acid ester, imino ester, carbodiimide anhydride and carbonate ester.

4. The composition of claim 3 wherein Z is N-hydroxysuccinimide.

5. The composition of claim 1 wherein X contains a radioactive isotope.

6. The composition of claim 5 wherein said radioactive isotope is selected from the group consisting of iodine, technetium, gadolinium, chromium and barium.

7. A method of imaging comprising administering a compound according to claim 1.

8. The method of imaging according to claim 7 wherein Z is selected from the group consisting of N-hydroxysuccinimide, N-hydroxy sulfosuccinimide, maleimide-benzoyl-succinimide, gamma-maleimido-butyryloxy succinimide ester, maleimidopropionic acid, isocyanate, thiolester, thionocarboxylic acid ester, imino ester, carbodiimide anhydride and carbonate ester.

9. The method of imaging according to claim 7 wherein X contains a radioactive isotope.

10. The method of imaging according to claim 9 wherein said radioactive isotope is selected from the group consisting of iodine, technetium, gadolinium, chromium and barium.

11. A method for non-invasively imaging an anatomical compartment of a mammalian host, comprising:
a) providing an imaging moiety, wherein said imaging moiety includes:
i) an anchor molecule having a carboxyl group; and
ii) a diagnostic agent;
b) reacting said imaging molecule with a chemical to convert said carboxyl group to a carboxylate ester group, thereby forming an activated imaging molecule, wherein said chemical is selected from the group consisting of: carbodiimides, phenols, thiophenols, benzyl alcohols and N-hydroxy imides;
c) administering said activated imaging molecule to the vascular system of said host;
d) forming in vivo at least one covalent bond between said carboxylate ester group and an amino, carboxyl or thiol group of said proteins; and
e) detecting said diagnostic agent.

12. A method according to claim 11 wherein said diagnostic agent is a biocompatible radioactive isotope.

13. A method according to claim 12 wherein said radioactive isotope is an element selected from the group consisting of iodine, technetium, gadolinium, chromium and barium.

14. A method for non-invasively imaging an anatomical compartment of a mammalian host, comprising:
a) providing an anchor molecule wherein said anchor molecule includes:
i) a carboxyl group, and
ii) a first binding member of a binding pair;
b) reacting said anchor molecule with a chemical to convert said carboxyl group to a carboxylate ester group, thereby forming an activated anchor molecule, wherein said chemical is selected from the group consisting of: carbodiimides, phenols, thiophenols, benzyl alcohols and N-hydroxy imides;
c) administering said activated anchor molecule to the vascular system of said host;
d) forming in vivo at least one covalent bond between said carboxylate ester group and an amino, carboxyl or thiol group of said proteins;
e) administering to the vascular system of said host a diagnostic compound wherein said diagnostic compound includes:
i) a second binding member of said binding pair, and
ii) a diagnostic agent;
f) forming said binding pair by binding said second binding member to said first binding member in vivo; and
g) detecting said diagnostic agent.

15. A method according to claim 14 wherein said first binding member is biotin and said second binding member is selected from the group consisting of avidin and streptavidin.

16. A method according to claim 14 wherein said diagnostic agent is a biocompatible radioactive isotope.

17. A method according to claim 16 wherein said radioactive isotope is an element selected from the group consisting of iodine, technetium, gadolinium, chromium and barium.

18. The method of claims 14 wherein said carboxyl group is N-hydroxysulfosuccinimide or N-hydroxysuccinimide.
